(19) United States
(12) Patent Application Publication
(10) Pub. No.: US 2008/0184574 A1
(43) Pub. Date: Aug. 7, 2008

(54) NECKLACE SIZER

(75) Inventor: George PRESS, New Vernon, NJ (US)

Correspondence Address: IP Patent Docketing;KIRKPATRICK & LOCKHART PRESTON GATES ELLIS LLP, 599 Lexington Avenue, 33rd Floor, New York, NY, 10022-6030, US

(73) Assignee: GEORGE PRESS INC., Livingston, NJ (US)

(21) Appl. No.: 11/670,184

(22) Filed: Feb. 1, 2007

(51) Int. Cl. A61B 5/103

(52) U.S. Cl. 33/512

(57) ABSTRACT

A necklace sizer useful for determining a necklace size for a prospective wearer employs a flexible measuring chain having a clasp at one end which is detachably attachable to the flexible measuring chain to provide a closed-loop measuring chain of selected length. The flexible measuring chain can be made up of a number of measuring links and at least one length-indicating tag. The measuring links, which can be ring-like are each of known length along the measuring chain and each measuring link can have the same length. The at least one length-indicating tag can be connected between and to each of two of the measuring links, can bear a length-indicating marking and can also have a predetermined length. Optionally, the at least one length-indicating tag can be apertured to receive the clasp. Useful embodiments of the necklace sizer can provide easily made and accurate size determinations. Some embodiments can simulate the look and feel of a necklace around the wearer's neck.

### STATEMENT REGARDING FEDERALLY SPONSORED RESEARCH OR DEVELOPMENT

(Not applicable.)

### BACKGROUND OF THE INVENTION

The present invention relates to a novel necklace sizer for determining a suitable necklace size for a prospective necklace wearer or other subject. The invention also relates to the use of the novel necklace sizer to supply an appropriately sized necklace to a prospective wearer.

The size of a necklace can affect the fit, feel and appearance of the necklace when it is worn. The same necklace can have a quite different look and feel on persons of different anatomies. The sizes appropriate for a slender child may be quite different from those appropriate for a large-bodied adult. One person may want a long pendulous necklace, another may want a short necklace that fits closely around the neck, while others may want something of intermediate length suitable for displaying a pendant ornament, symbol, jewel, image or the like. Still other prospective customers or wearers may want their necklace to have multiple strands in one or another style.

Accordingly, necklaces can be, and often are, available in a range of sizes. To speed selection and reduce the handling of what may be expensive items, a necklace sizer which can help determine an appropriate size for a prospective wearer can be helpful. As an alternative to selecting from a range, a particular necklace may be custom fitted and supplied to the specific size requirements of an individual wearer. Custom fitting a necklace is likely to require a significant investment of time and materials on the part of the supplier. In some cases the product may be wearer-specific and it is then of limited value to other prospective wearers, or customers, should the customer be dissatisfied with the length of the customized product. Again, a necklace sizer can be useful to prevent or mitigate this potential problem.

It is relatively easy to determine the size of a prospective wearer's neck. This can be done with an ordinary flexible tape measure, such as is used for sewing, by a fitter wrapping the tape measure around the prospective wearer's neck and reading off a length. However, in some cases, even this simple measurement may raise questions of style and location of the measurement. For example, the measurement could be made around the narrowest part of the base of the neck. Alternatively, especially where the base of the neck tapers, the measurement can be made at a lower, wider point. For looser fits, for example, where a necklace is to depend on to a wearer's torso, a tape measure may be unsuitable for accurate sizing because it is reluctant to adopt the form and lie, or drape, of a necklace making accurate measurements difficult to obtain.

Various proposals have been made for sizing articles other than necklaces to fit the human body, but none appears suitable for use as a necklace sizer.

For example, U.S. Pat. No. 3,639,995 to Gardner discloses a device for measuring the size of a finger to determine the size of a ring that will fit same. A chain carried in a casing is pulled into a loop outside the casing into which loop the finger to be measured is inserted. A movable pointer indicates the ring size on a scale on the device.

U.S. Pat. No. 5,920,998 to Slilaty discloses a tape measuring system and a combination collar-and-tape measuring tool. The latter comprises a portion of a garment collar to which a tape measure is attached. The inside of the tape measure has a lining of material which can attach to a mating surface on the collar portion.

U.S. Pat. No. 7,058,983 to Namiki discloses a necktie containing a chain-like winding portion that is wound around the neck. The winding portion bears a clasp at one end which is attachable to, and removable, from any portion of the winding portion.

In addition, a necklace sizer has been proposed which comprises a chain of twisted-loop links from which a number of length-labeled marker tags hang at approximate distance intervals along the chain. However, this device, which is described in more detail below, lacks adequate accuracy and may be difficult to use. Accordingly, there is a need for a necklace sizer which is accurate and convenient to use.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the present invention.

### BRIEF SUMMARY OF THE INVENTION

The present invention provides, in one aspect, a necklace sizer for determining a necklace size for a prospective wearer. The necklace sizer can comprise a flexible measuring chain intended to be disposed around the neck of the prospective wearer, and the measuring chain can comprise a number of measuring links as well as at least one length-indicating tag. Each measuring link can have a predetermined length along the measuring chain. Also, the at least one length-indicating tag can have a predetermined length along the measuring chain and this length can be the same as, or different from, the length along the measuring chain of each measuring link. The at least one length-indicating tag can be disposed between, and connected to, each of two of the measuring links. The necklace sizer can further comprise a releasable clasp fixed to the measuring chain. The clasp can be selectively and detachably attachable to the measuring links, or to the at least one length-indicating tag, to provide a closed-loop measuring chain of selected length.

In some embodiments of the invention, the measuring links each have the same predetermined length along the measuring chain. If desired, the or each length-indicating tag can have the same predetermined length along the measuring chain as each measuring link. The, or each, length-indicating tag can comprise an opening to receive and locate the releasable clasp. The releasable clasp can be insertable into the opening and be detachably attachable to the or each length-indicating tag.

The necklace sizer can comprise multiple length-indicating tags and the tags can be equi-spaced along the measuring chain. If desired the tags can have an approximately laminar shape, for example the shape of a disk. The or each length-indicating tag can comprise a marking to indicate a distance along the measuring chain from the releasable clasp to the length-indicating tag.

Each individual measuring link can comprise a closed loop extending through at least one other link or tag and can, for example, comprise an approximately circular ring. In this, or another way, each measuring link can define an opening. The releasable clasp can have a resiliently biased latch member receivable into each measuring link opening.

The flexible measuring chain can have two ends and the releasable clasp can be secured to one of the two ends of the measuring chain.

In another aspect, the invention provides a method of using the necklace sizer for supplying a necklace to a prospective wearer. The method generally comprises fitting the necklace sizer around the neck of the prospective wearer, or other subject, for example a person buying a necklace as a gift. Furthermore, the method can comprise engaging the releasable clasp in a selected measuring link or marker tag to form a closed-loop of selected length around the neck of the prospective wearer or other subject. The selected length of the necklace sizer can then be read off by reference to the length-indicating tag in the closed loop portion of the necklace sizer that is closest to the releasable clasp and, if desired, by also counting off any intervening measuring links. The method can include supplying a necklace of the determined size to the prospective wearer.

Useful embodiments of the necklace sizer of the invention can provide easily made and accurate size determinations. Some embodiments can simulate the look and feel of a necklace around the wearer's neck.

### BRIEF DESCRIPTION OF THE SEVERAL VIEWS OF THE DRAWING

Some embodiments of the invention, and of making and using the invention, as well as the best mode contemplated of carrying out the invention, are described in detail herein, by way of example, with reference to the accompanying drawings, in which like reference characters designate like elements throughout the several views, and in which:

FIG. 1 is a perspective view of a known necklace sizer;

FIG. 2 is an enlarged perspective view of a portion of the known necklace sizer shown in FIG. 1; and

FIG. 3 is a perspective view of an embodiment of necklace sizer according to the invention.

### DETAILED DESCRIPTION OF THE INVENTION

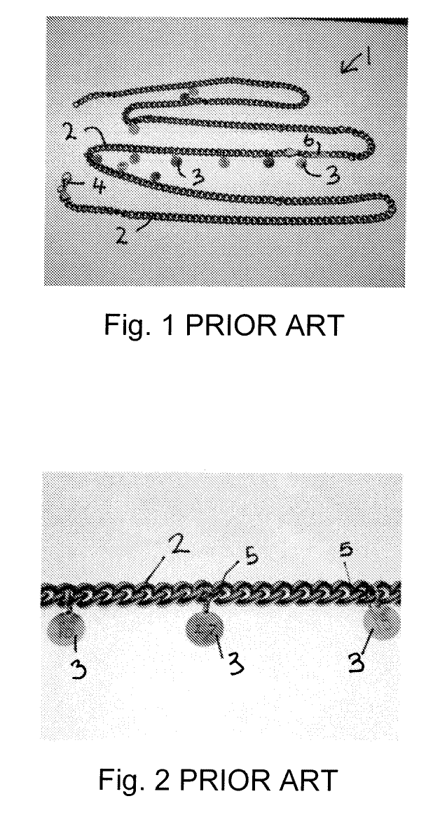

Referring to FIGS. 1 and 2, the known necklace sizer illustrated, referenced 1, comprises a chain 2 formed of twisted metal links from which hang a number of length-labeled marker tags 3. Marker tags 3 are each attached to chain 2 by a small ring 5 which passes through one of the links. Marker tags 3 can be positioned at unit distances and suitably labeled, for example with labels for 16, 17 and 18 inches of length (about 406, 432 and 457 mm, respectively), as shown in FIG. 1. A resiliently biased clasp 4 attached to one end of necklace sizer 1 can be releasably attached to chain 2 at a selected position to fit necklace sizer 1 to a prospective wearer. Clasp 4 grips chain 2 by clamping on to one or two links 2. The clamping position can be varied more or less continuously along the chain. However the uneven profile provided by the chain links makes accurate positioning difficult. Necklace sizer can be fabricated from a metal such as brass.

With clasp 4 suitably positioned, the selected length of necklace sizer 1 can be read off the marker 3 that is nearest to clasp 4. Subunit distances can be approximately estimated by counting off the number of links, if any, between clasp 4 and the nearest marker 3. The measurement obtained is a useful general indication of possible necklace length, but may not be sufficiently accurate. As can be seen in FIG. 1, the number of links between marker tags 3 can vary. As shown, there are seven links between the 16-inch marker tag 3 and the 17-inch marker tag 3 and eight links between the 17-inch marker tag 3 and the 18-inch marker tag 3. Thus, it can be difficult to locate marker tags 3 at precise intervals of length. Furthermore the presence of ring 5 in the opening of the respective link 2 can make it difficult to attach clasp 4 at the same point on chain 2, introducing a further potential inaccuracy.

As may be seen from FIG. 1, necklace sizer 1 is formed in two separable sections joined by a connector 6, providing a choice of lengths of the necklace sizer.

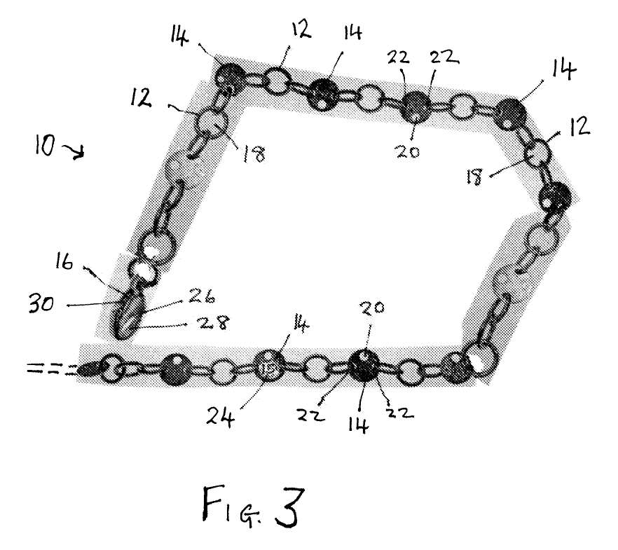

Referring now to FIG. 3, the illustrated necklace sizer embodying the invention, which is referenced 10, comprises a measuring chain formed of a linear string of interconnected measuring links 12 and of length-indicating marker tags 14. In the illustrated embodiment, each measuring link 12 and marker tag 14 has a predetermined length along necklace sizer 10 when the latter is fully extended. However, other embodiments are possible, as will be apparent from this disclosure. If desired, necklace sizer 10 can be calibrated for accurate length measurement. Calibration can be facilitated, for example, by providing a construction wherein marker tags can be precisely located at discrete length increments, as described herein. Additionally, calibration can be facilitated by fabricating each of the components of the measuring chain to have a lengths along the measuring chain which are precise units of measurement and/or simple subunits of those units of measurement. Some examples of possible units and subunits are inch units and quarter- or eighth-inch subunits. Other examples are centimeter units and 5 mm or 2 mm subunits. Necklace sizer 10 can be calibrated by extending it and comparing the extend device with a length standard, for example, a high quality measuring tape. If necessary, adjustments can be made by replacing or modifying defective measuring links 12 or marker tags 14, for example by deforming or machining, so that marker tags 14 are properly positioned at appropriate length intervals. Marker tags 14 can then be marked to display or otherwise indicate appropriate lengths along the necklace sizer.

At one end, necklace sizer 10 comprises a releasable clasp 16 which can be detachably engaged with any one of measuring links 12 or any marker tag 14 to form a closed-ended loop. The other end of necklace sizer 10 can comprise a simple link 12, a marker tag 14 or other desired termination (not shown).

Each measuring link 12 comprises a closed loop of suitable material such as metal wire or rod, molded plastic, ceramic or other material. In some embodiments of the invention each measuring link 12 is formed of a relatively dense material which remains substantially rigid during normal use and which has an attractive appearance. An attractive feel can also be desirable. For example each measuring link 12 can be formed of silver, brass, bronze, aluminum, a silver alloy, gold, a gold alloy, faux gold or the like. If desired, each measuring link 12 can be completely or partially covered or coated in a clear or opaque plastics material. Such a plastics covering or coating can enhance the feel of the measuring chain on the neck or torso of a prospective wearer, avoiding a cold feeling.

Desirably, each measuring link 12 has a construction such as to have a predictable length along the measuring chain when the measuring chain is fully extended or taut. To this end, a given measuring link 12 can be approximately circular, as shown in FIG. 3, so that the chain length subtended by the link when necklace sizer 10 is extended, is independent of the orientation of the link.

Other measuring link shapes can also be employed, provided that the shapes enables the link to adopt a predictable configuration and the link will subtend a predetermined length along the extended necklace sizer, facilitating accurate measurement. Such shapes include oval, elliptical, elongated and parallel-sided links, “anchor chain” links having part-circular ends, square, diamond-shaped, star shaped links and other link configurations as will be known or apparent to those skilled in the art.

As shown in FIG. 3, each measuring link 12 can have a central opening 18 through which one or both neighboring measuring links 12 pass. Central opening 18 can receive releasable clasp 16 or a component thereof.

Alternatively, in other embodiments of the invention (not shown), each link 12 can have a solid or filled center and can have two separate apertures, or openings, to accommodate neighboring links 12 and can have a further aperture, opening or recess to receive releasable clasp 16.

In some embodiments of the invention, all the measuring links 12 in the measuring chain are identical or substantially identical, differing only in minor respects such as coloring, ornamentation or the like, so that each measuring link 12 provides a single unit of length along the measuring chain. In other embodiments of the invention, measuring links 12 of two or more different characters, for example circular links and elongated links are employed in the same measuring chain. Each type of measuring link 12 can have the same unit length along the measuring chain. Alternatively, the different types may have different lengths, optionally in a simple ratio one to the other. For example, in the just-referenced alternative embodiment, the elongated links can be twice as long as the circular links. Suitable combinations of different-sized measuring links 12 can be employed to give necklace sizer 10 desired graduations, or intervals, along its length.

In the illustrated embodiment of the invention, marker tags 14 each comprise a circular, flat or planar, and laminar disk, which resembles a coin or a token. Each marker tag 14 has an opening 20 to receive and locate releasable clasp 16 and two apertures 22 through each of which an adjacent or neighboring measuring link 12 passes. Releasable clasp 16 is insertable into or engageable with each opening 20 to be detachably attachable to the or each marker tag 14.

Desirably, opening 20 can be a close fit to the received component of releasable clasp 16 and is positioned on marker tag 14 in a location selected to provide a predetermined, or unit, displacement along necklace sizer 10 when releasable clasp 16 is engaged, to facilitate accurate size determinations. For example, opening 20 can be located at a midpoint between apertures 22 so that the centers of opening 20 and apertures 22 lie approximately on an isosceles triangle. If desired, a second opening 20 (not shown in FIG. 3) engageable by releasable clasp 16 can be provided in one or more marker tags 14, and the second opening 20 can be disposed on the other side of marker tag 14 from apertures 22. Desirably also, apertures 22 are so positioned, and the size of each marker tag along necklace sizer 10 is selected to provide a specific displacement along necklace sizer 10 when the latter is extended. Each marker tag 14 can have the same predetermined length along the measuring chain as one or more of the measuring links 12. In one embodiment of the invention all the measuring links 12 and the marker tags 14 have the same length.

So long as the shape will accommodate the spatial or geometric requirements of their measuring roles, each marker tag 14 can have any desired shape as an alternative to the disc-like shape shown in FIG. 3. For example, marker tags 14 can have other shapes including triangular, square, rectangular, hexagonal, polygonal or a fanciful representation of a jewel, character or other representative shape. Marker tags 14 can have a relatively flat or laminar form or can have a three-dimensional shape, optionally with a flat or other display surface. For example, marker tags 14 can have a rounded shape, a spherical or part-spherical shape, cubic, representational or other shape. In one embodiment of the invention, marker tags 14 have the shape of a cut diamond or other jewel, and optionally have a display surface for the display of indicia.

Marker tags 14 can each be the same as one another or they may be different, one from another. Marker tags 14 can be equi-spaced along necklace sizer 10, or can have other desired spacing. As shown, in the illustrated embodiment, marker tags 14 are attached to necklace sizer 10 at regular intervals to mark distances along the length of necklace sizer 10.

In some embodiments of the invention, marker tags 14 are spaced at specific unit distances along necklace sizer 10, for example at one-inch, or ten- or twenty-millimeter intervals. Measuring links 12 can have lengths which are uniform sub-divisions of the unit distance, for example a quarter or a fifth of the unit. Desirably, in such an embodiment of the invention, the lengths of marker tags 14 are equal to the sub-division. Thus marker tags 14 and measuring links 12 cooperate to enable clasp 16 to be engaged at exact unit or subunit distances along the measuring chain which distances can be predetermined with good accuracy. Clasp 16 can be easily and accurately located in a measuring link opening 18 or a marker tag opening 20, as will be further described. Thus, necklace sizer 10 can be calibrated or indexed in precise increments. The increments can be chosen to be appropriate to the necklace to be supplied and may for example be intervals of one-quarter inch, one-half inch, 6 mm or 10 mm or other desired length.

In one embodiment of the invention, measuring links 12 and marker tags 14 have a length of one-quarter inch (about 6 mm) and measuring links 12 and marker tags 14 are arranged in a sequence of repeats of three measuring links 12 followed by a marker tag 14.

Marker tags 14 can bear visible indicia such as is illustrated by reference 24, for example numbers, to indicate distance along necklace sizer 10 in inches, millimeters or other desired units. If desired, marker tags 14 can be labeled in numerical sequence beginning with the marker tag closest to releasable clasp 16 and continuing along necklace sizer 10 according to its length. For example marker tags 14 can be numbered from 1 to 18, 1 to 24 or 1 to 36 to indicate neck size graduations in inches up to 18, 24 and 36 inches respectively.

Since very short necklace lengths, such as say 8 inches (about 200 mm) or less are unlikely to be useful, some of the marker tags closer to releasable clasp 16 can be unmarked, if desired, or even omitted and replaced by appropriately sized measuring links 12 or other spacers. Alternatively, the portion of necklace sizer close to releasable clasp 16, which is too short to have a useful necklace length, for example, 10 inches (about 250 mm) or 8 inches (about 200 mm) or less, can be a stock length of predetermined measurement of flexible cord, chain, wire, or the like.

In further embodiments of necklace sizer 10, marker tags 14 can visually indicate length along the necklace sizer simply by their presence at known distances along necklace sizer 10. The visual distinctiveness of marker tags 14 enables distances along necklace sizer 10 readily to be determined by counting off marker tags 14 along necklace sizer 10. Alternatively, marker tags 14 can have different colors or shapes or colors and shapes to indicate length along necklace sizer 10, if desired.

If desired, some, or even all, measuring links 12 can also be numerically labeled to indicate unit subdivisions between marker tags. However, if the spacing of marker tags 14 is relatively frequent so that there are only a small number of measuring links 12 between successive marker tags 14, for example three or four, it will be easy for a user to visually determine fractional lengths between marker tags 14.

As shown in FIG. 3, releasable clasp 16 can, as shown, be of a “lobster claw” style and can comprise a spring-biased movable latch member 26 which is engageable with an opposed claw 28 and manually actuated by a push-button 30. Usefully, movable latch member 26 can be received into opening 18 of a measuring link 12 or opening 20 of a marker tag 14 to attach clasp 16 to the necklace sizer chain. Releasable clasp 16 can be attached to the chain of measuring links 12 and marker tags 14 in any suitable manner, with or without other fittings, for example by employing a ring 32. Ring 32 and releasable clasp 16, considered together, desirably can have a specific length along the measuring chain, which can be a unit or subunit of length, for example, an inch or one or two centimeters.

Releasable clasp 16 can have any other suitable structure. For example, releasable clasp 16 can comprise a crocodile clamp, in which case measuring links 12 and marker tags 14 may not need openings to receive releasable clasp 16. Alternatively, ridges or other abutments, recesses or the like, can be provided to locate a crocodile-clamp releasable clasp 16. In another alternative, releasable clasp 16 can comprise a simple hook. Other chain attachment devices that can be readily manipulated to be attached to and detached from a measuring link 12 or marker tag 14, and which have fixed and specific length characteristics, can be employed in place of clasp 16, if desired.

For accuracy, releasable clasp 16 or other chain attachment device, if employed, and any fittings or the like employed to attach same to the chain of measuring links 12 and marker tags 14 desirably can have a known fixed length along the measuring chain. For example they can have a length equal to a measuring unit or to a subunit or to a convenient multiple of subunit lengths.

As stated previously, the other end of necklace sizer 10 from clasp 16 can terminate with a measuring link 12, a marker tag 14 or another termination device. Such other termination device can for example be a plaque or medallion or a grip. The termination device that can be plain or, if desired, can bear indicia or ornamentation, for example the name, trademark or logo of a vendor, supplier or other party. Alternatively, the termination device can comprise a small cross-bar or other device facilitating manipulation of the necklace sizer end.

If desired, necklace sizer 10 can be modular comprising one or more removable chain sections (not shown) which are effective to vary the length of the necklace sizer. Each chain section can bear a small terminal clasp to removably attach the chain section to necklace sizer 10 to increase its length. To maintain the measuring characteristics, the clasp can have a dimension along the necklace sizer which is a predetermined unit or subunit of length, for example the length of one, or possibly two or more, chain links 12.

The resultant sectionalized and variable length necklace sizer can be employed in different length for different purposes, if desired. For example, a short necklace sizer can be employed to size a prospective wearer for a choker or other close-fitting necklace or the like, or to size children. One or more chain sections can then be added to the short necklace sizer to size for an adult or for a longer necklace such as a pendant or a continuous two or three strand neck lace.

One possible use of necklace sizer 10 will now be described by reference to an embodiment comprising multiple marker tags 14. Other uses and ways of using other embodiments of a necklace sizer according to the invention will be apparent, or become apparent to those skilled in the art in light of this disclosure.

In use, necklace sizer 10 can be draped or fitted around a prospective wearer's neck by a fitter. The necklace fitter, while holding the measuring chain of necklace sizer 10 in one hand and grasping releasable clasp 16 in the other, can select a suitable measuring link 12, or marker tag 14 in which to engage releasable clasp 16 to form a closed-loop, necklace-like chain, of selected length, around the prospective wearer's neck. The fitter and prospective wearer can converse about a desirable length for the contemplated necklace by reference to the selected length of the necklace sizer. The fitter can readily adjust the necklace sizer length for comfort or style, for example choker, high pendant, low pendant or double or triple strand, or other desired style, by moving releasable clasp 16 to a different measuring link 12 or marker tag 14 to give necklace sizer 10 a different length.

If desired, the length of necklace sizer 10 can be adjusted to provide a particular drape desired by the prospective wearer or other subject. The drape of the necklace sizer can, for example, simulate how a necklace may conform to, and/or flow across the wearer's anatomy or attire or both.

Once a satisfactory fit and style have been determined, with the assistance of a mirror, if desired, the fitter can conveniently read off the length of necklace sizer 10 that has been selected. The length can be read by referring to the marking on the marker tag 14 in the closed loop portion of necklace sizer 10 that is closest to releasable clasp 16 and by counting off any intervening measuring links 12. Engagement of clasp 16 in central opening 18 of a measuring link 12 or of opening 20 in a marker tag 14, is effective to hold the length measurement while the measurement is read, allowing necklace sizer 10 to be moved or oriented to a good viewing position, if desired, without disturbing the length measurement.

Alternatively, the prospective wearer, or other person can place necklace sizer 10 around their own neck using a mirror, if desired, to assist a determination of a desired fit style and can make the size determination without the assistance of a fitter.

By suitable selection of its dimensions, as will be apparent to one skilled in the art, materials and components, necklace sizer 10 can simulate the drape and feel of a necklace, following the wearer's contours and giving an approximate simulation of how a contemplated necklace may look and feel.

If necklace sizer 10 is sectionalized, as described herein, the fitter can select a base section to size a choker, a child or other application for a relatively short necklace. When a longer necklace is required, for other applications, the fitter can add one or more chain sections.

The invention includes a method of supplying a necklace to a customer, or other prospective wearer comprising selecting a necklace sizer according to the invention that is appropriate for a necklace style desired by the prospective wearer. The method additionally comprises using the selected necklace sizer to determine a suitable necklace size for the prospective wearer, for example by determining a suitable fit and style in the manner described herein and supplying a necklace of the determined size. The sized necklace can be selected from a range of stock sizes, or can be custom fabricated. Some examples of necklaces that can be supplied in this manner include gold, silver and platinum chains and single-, double- and triple-strand pearl necklaces. Other examples will be apparent to those skilled in the art in light of this disclosure.

Embodiments of the invention are suitable for use or performance in a jewelry or department store or other location where necklaces or the like are available. If desired, low cost embodiments of necklace sizer 10 can be supplied to prospective customers, or wearers, for their personal use, or to facilitate mail order or internet sales.

It will be understood that a necklace sizer according to the invention can also be employed to size collars or other products that are worn around the neck, or may be used for other sizing purposes for example as a bracelet sizer or an anklet sizer. If desired, a necklace sizer according to the invention can be furnished in a suitable length to serve as a bracelet sizer, an anklet sizer or another desired sizer. In an alternative embodiment, the invention provides a multipurpose necklace sizer which has one or more removable sections to enable it to also serve as a bracelet sizer, anklet sizer or the like. The sections can be releasably attachable to one another by suitable fasteners, for example one or more lobster claws. For example a necklace sizer having a length of about 30 inches (about 750 mm) can have a removable section of about 18 or about 20 inches (about 450 or about 500 mm). The removable section can have a lobster claw or comparable clasp at each end and can be removed to enable the necklace sizer to serve as a bracelet sizer.

Throughout the description, where compositions or structures are described as having, including, or comprising specific components, or where processes are described as having, including, or comprising specific process steps, it is contemplated that compositions or structures of the present invention may also consist essentially of, or consist of, the recited components, and that processes embodying the present invention may also consist essentially of, or may consist of, the recited processing steps. It should be understood that the order of steps, or order for performing certain actions, is immaterial provided that the respective process embodiment is useful for the purposes of the invention. Moreover, two or more steps or actions may be conducted simultaneously, if the context permits.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art. Should there appear to be conflict between the meaning of a term as used in the written description of the invention in this specification and the usage in material incorporated by reference from another document, the meaning as used herein is intended to prevail.

While illustrative embodiments of the invention have been described above, it is, of course, understood that many and various modifications will be apparent to those of ordinary skill in the relevant art, or may become apparent as the art develops, in the light of the foregoing description. Such modifications are contemplated as being within the spirit and scope of the invention or inventions disclosed in this specification.

1. A necklace sizer for determining a necklace size for a prospective wearer, the necklace sizer comprising a flexible measuring chain to be disposed around the neck of the prospective wearer, the flexible measuring chain comprising:
(a) a number of measuring links, each measuring link having a predetermined length along the measuring chain,
(b) at least one length-indicating tag disposed between and connected to each of two of the measuring links, the at least one length-indicating tag having a predetermined length along the measuring chain being the same as or different from the length along the measuring chain of each measuring link; and
(c) a releasable clasp fixed to the measuring chain, the clasp being selectively and detachably attachable to the measuring links or the at least one length-indicating tag to provide a closed-loop measuring chain of selected length.

2. A necklace sizer according to claim 1 wherein the measuring links each have the same predetermined length along the measuring chain.

3. A necklace sizer according to claim 2 wherein the or each length-indicating tag has the same predetermined length along the measuring chain as each measuring link.

4. A necklace sizer according to claim 1 wherein the or each length-indicating tag comprises an opening to receive and locate the releasable clasp, the releasable clasp being insertable into the opening and being detachably attachable to the or each length-indicating tag.

5. A necklace sizer according to claim 1 comprising multiple said length-indicating tags equi-spaced along the measuring chain.

6. A necklace sizer according to claim 1 wherein the or each length-indicating tag has an approximately laminar shape.

7. A necklace sizer according to claim 1 wherein the or each length-indicating tag comprises a disk.

8. A necklace sizer according to claim 1 wherein the or each length-indicating tag comprises a marking to indicate a distance along the measuring chain from the releasable clasp to the length-indicating tag.

9. A necklace sizer according to claim 1 wherein each individual link comprises a closed loop extending through at least one other link.

10. A necklace sizer according to claim 1 wherein each link comprises an approximately circular ring.

11. A necklace sizer according to claim 1 wherein each measuring link defines an opening and the releasable clasp comprises a resiliently biased latch member receivable into each measuring link opening.

12. A necklace sizer according to claim 1 wherein the flexible measuring chain has two ends and wherein the releasable clasp is secured to one of the two ends of the measuring chain.

13. A necklace sizer according to claim 1 comprising multiple said length-indicating tags equi-spaced along the measuring chain wherein the measuring links and the length-indicating tags each have the same predetermined length along the measuring chain, wherein the length-indicating tags each comprise an opening to receive and locate the releasable clasp and wherein the releasable clasp is insertable into the opening and is detachably attachable to the or each length-indicating tag.

14. A necklace sizer according to claim 1 wherein the or each length-indicating tag comprises a marking to indicate a distance along the measuring chain from the releasable clasp to the tag and optionally comprises a disk, wherein each individual link comprises a closed loop extending through at least one other link and defining an opening, wherein the releasable clasp comprises a resiliently biased latch member receivable into each measuring link opening and wherein the measuring chain has two ends, the releasable clasp being secured to one of the two ends of the measuring chain.

15. A necklace sizer according to claim 14 wherein the closed loop comprised by each individual link comprises an approximately circular ring.

16. A necklace sizer according to claim 13 wherein the or each length-indicating tag comprises a marking to indicate a distance along the measuring chain from the releasable clasp to the tag and optionally comprises a disk, wherein each individual link comprises a closed loop extending through at least one other link and defining an opening, wherein the releasable clasp has a resiliently biased latch member receivable into each measuring link opening and wherein the measuring chain has two ends, the releasable clasp being secured to one of the two ends of the measuring chain.

17. A necklace sizer according to claim 16 wherein the closed loop of each individual measuring link comprises an approximately circular ring.

18. A method of using a necklace sizer according to claim 1 for supplying a necklace for a prospective wearer, the method comprising placing the necklace sizer around the neck of a subject, selecting a suitable measuring link or length-indicating tag to provide a suitable, engaging the releasable clasp in a selected measuring link or length-indicating tag to form a closed-loop of selected length around the neck of the subject, reading the selected length of the necklace sizer by reference to a length-indicating tag in the closed loop portion of the necklace sizer, being the length-indicating tag closest to the releasable clasp.

19. A method according to claim 18 comprising supplying a necklace of the determined size to the prospective wearer.

20. A method according to claim 19 wherein the reading of the selected length further comprises counting off any measuring links intervening between the length-indicating tag closest to the releasable clasp and the releasable clasp.
